(19) United States
(12) Patent Application Publication
(10) Pub. No.: US 2015/0223752 A1
(43) Pub. Date: Aug. 13, 2015

(54) Cardiac Emergency Response Facilitation Method and System

(71) Applicants: Michael Joseph Kurtz, Philadelphia, PA (US); Jorge Cesmegi, III, Willow Grove, PA (US); Patrick Moran, Philadelphia, PA (US)

(72) Inventors: Michael Joseph Kurtz, Philadelphia, PA (US); Jorge Cesmegi, III, Willow Grove, PA (US); Patrick Moran, Philadelphia, PA (US)

(21) Appl. No.: 14/178,164

(22) Filed: Feb. 11, 2014

(51) Int. Cl. A61B 5/00; G06N 5/04; G09B 23/28; G06F 19/00; G09B 5/00; A61N 1/39

(52) U.S. Cl. CPC A61B 5/4839; A61B 5/7264; G09B 5/00; A61N 1/3987; G09B 23/285; G06F 19/345; G06N 5/04

(57) ABSTRACT

In an embodiment of the disclosed technology, a method and system are used for assisting and facilitating cardiac emergency treatment. The method employs an electronic interface for logging the different treatments and/or medicines administered to a cardiac emergency patient. The interface maintains numerous time intervals, dosage frequencies, and dosage amounts for different treatments. The interface prompts the user when to administer different treatments based on the type of cardiac emergency being suffered, as well other factors. The device implementing the electronic interface may further be in communication with treatment mechanisms, such as a defibrillator and/or an automatic dosage administration machine, for purposes of automated application of treatment at the prompts. Patient information and history, including the history from the present treatment regimen, is stored in a database accessible via the interface.

### FIELD OF THE DISCLOSED TECHNOLOGY

The disclosed technology relates generally to medical emergency response and, more specifically, to a system for facilitating and monitoring patient care during a cardiac emergency.

### BACKGROUND OF THE DISCLOSED TECHNOLOGY

Cardiac arrest, heart attacks and other heart failures are major causes of death and serious injury. Each day thousands of Americans suffer cardiac emergencies. Cardiac emergencies typically strike without warning, often affecting people with no history of heart disease. Because of the potentially life-threatening nature of cardiac emergencies, it is imperative that victims receive immediate care to prevent secondary, permanent, damage to the brain or, worse yet, death.

Cardiac emergencies include: acute myocardial infarction (commonly referred to as “heart attacks”); bradycardia; tachycardia; hypotension and pulmonary edema; ventricular fibrillation (“VF”) and ventricular tachycardia (“VT”); pulseless electrical activity (“PEA”); and asystole. Each cardiac emergency has to follow its own treatment protocol which is determined by the specific symptoms manifested by the victim. One of the most common cardiac emergencies is sudden cardiac arrest (“SCA”). It is estimated that more than one thousand people per day are victims of sudden cardiac arrest in the United States alone.

Because the heart may no longer be pumping blood effectively during a cardiac emergency, the chances of surviving decrease with time elapsed after the onset of the emergency. Brain damage can occur after the brain is deprived of oxygen for four to six minutes. Often physicians are unable to attend to cardiac emergency patients until the patient is brought into a hospital. Thus, many patients suffering from cardiac emergencies are at first treated by nurses and EMT responders. These first responders usually do not have the depth of experience and training in treating cardiac emergencies that physicians do.

Furthermore, when a cardiac emergency patient receives treatment outside of a hospital or ambulance, the person rendering this treatment may not have all of the resources that a physician or nurse may have within the confines of an ambulance or hospital. Such resources may include medical devices, books, and digitized information for assisting in the treatment of the patient.

CPR (cardiopulmonary resuscitation) is a combination of artificial respiration (“rescue breathing” or “expired air resuscitation”) and artificial circulation (“external cardiac compression” or “external chest compression”). Typically, if the patient is unconscious and is not breathing, but has a pulse, rescue breathing only is required. Whereas, if the patient is unconscious, is not breathing, and has no pulse, rescue breathing along with external cardiac compression is required.

Rescue breathing is performed by first clearing and opening the air passage. Once the airway is cleared, if the patient is still unable to breathe, the rescuer pinches the nose of the patient and slowly breathes into the mouth of the patient until the patient's chest rises. Additionally, a barrier mask, bag-valve mask, automatic transport ventilators (“ATVs”), or oxygen-powered, manually triggered devices may be used by the rescuer during rescue breathing, in order to protect the rescuer from direct contact with the patient's bodily fluids. According to current AHA guidelines, the patient should be ventilated by rescue breathing twice before performing the remaining steps of CPR. Once the patient has been ventilated twice, the patient's pulse is checked. If a pulse is present, and the patient's breathing has not resumed on its own, then the rescue breathing procedure should be continued.

Treatment of patients experiencing cardiac arrest and other emergencies is often very regimented. Specific routines are followed by physicians regarding dosage and time intervals for treatments. Because rapid response to a cardiac arrest is critically important, the American Heart Association (AHA) developed the “Chain of Survival” guidelines, which recite the following steps: 1. Early access to an emergency medical service (EMS), such as by activating an emergency response system (e.g., calling an ambulance or dialing “911”); 2. early CPR initiated by a bystander or other early caregiver to help the patient survive until more advanced care becomes available; 3. early defibrillation; and 4. early application of Advanced Cardiac Life Support (ACLS), such as airway management, drugs, etc. The benefits of this approach to survival are discussed in more detail in Cummins, et al., “Improving Survival from Sudden Cardiac Arrest: the ‘Chain of Survival’ Concept” Circulation 83:1832-1847 (1991). With the exception of the defibrillation step (#3.), these guidelines are appropriate for treating victims of all cardiac emergencies, not just SCA. Administration of cardiac medications such as epinephrine and isoproterenol may not be so straightforward. However, if the regimen is followed properly and treatment is rendered immediately, such medications may save a patient's life.

### SUMMARY OF THE DISCLOSED TECHNOLOGY

Therefore, it is an object of the disclosed technology to provide a method and system for guiding the administration and facilitation of cardiac emergency medicine to a patient. The technology uses a plurality of timers configured based on various medically-accepted dosage frequencies and amounts of drugs used in treating cardiac emergencies.

As such, in an embodiment of the disclosed technology, a method is used for assisting and facilitating cardiac emergency treatment. The method is carried out, not necessarily in the following order, by: a) receiving an input from a medically trained user classifying the type of cardiac emergency being experienced by a patient; b) prompting the user to administer to the patient a first dosage of a first drug based on the inputted cardiac emergency classification; c) receiving an input confirming the administration of the first dosage of the first drugs; d) prompting the user to re-administer the first drug after a pre-specified time has elapsed; e) receiving an input from the user about defibrillation parameters used in a first defibrillation applied to the patient, the defibrillation parameters being defibrillation mode and amount of power; and/or f) prompting the user to re-defibrillate after a specific time interval. In embodiments, the specific time interval may be based on the inputted cardiac emergency classification and the defibrillation parameters.

A “cardiac emergency” is defined as the onset of any type of heart condition that may result in injury or death. Cardiac emergencies may include cardiopulmonary arrest (commonly referred to as “cardiac arrest”), acute myocardial infarction (commonly referred to as “heart attacks”); bradycardia; tachycardia; hypotension and pulmonary edema; ventricular fibrillation (VF) and ventricular tachycardia (VT); pulseless electrical activity (PEA); and asystole.

The method may have an additional step of receiving input information regarding patient characteristics, and using the patient characteristics to determine treatment parameters. The patient characteristics may refer to age, weight, height, and/or medications,

In a further embodiment, the defibrillator is connected to the interface, and the interface triggers each defibrillation at the specified time intervals. All of the data inputted regarding treatment may be logged and stored for a given patient. The logged data may be used in rendering future treatment to the patient and/or to other patients.

In another embodiment of the disclosed technology, a system uses artificial intelligence to facilitate treatment on a patient experiencing a cardiac emergency. The system may employ one or more of the following components: a) a data processing apparatus; b) a computer storage device storing instructions that, when executed by data processing apparatus, cause the data processing apparatus to perform operations; c) an interactive interface for inputting real-time patient data and displaying treatment prompts based on the real-time patient data; d) a database for logging patient data and treatments administered; and/or e) artificial intelligence to modify treatment prompts based on inputted real-time patient data. “Artificial intelligence,” for purposes of this specification, is defined as a machine, computer and/or software, that are capable of reasoning, retaining knowledge, planning, learning, communication, decision-making, and perception.

In further embodiments, the inputted patient data are received electronically from sensors associated with the patient. The system may further employ a defibrillator electronically coupled to the data processing apparatus wherein the defibrillator is controlled by the interface. The system may also employ an automated dosing machine electronically coupled to the data processing apparatus for administering medicine to the patient via the interface.

In still another embodiment of the disclosed technology, a method uses artificial intelligence for determining and prescribing a treatment regimen for a patient suffering a cardiac emergency. The method is carried out, not necessarily in the following order, by: a) receiving inputted symptom data observed from the patient; b) receiving inputted biological data regarding the patient; c) diagnosing the cardiac emergency suffered by the patient using artificial intelligence; d) prescribing a protocol for treating the patient; e) displaying, on a device, prompts outlining parameters for a treatment to be administered to the patient; f) receiving and logging inputted treatment data regarding the treatment administered to the patient; and/or g) modifying the parameters based on the inputted treatment data.

In embodiments, the treatment may be defibrillation. and the parameters may be defibrillation mode, power and time between shocks. “Defibrillation mode” is defined as the classification of the shock delivered (typically biphasic, monophasic, or lown). “Power” is defined as the charge (typically in joules) delivered to the patient.

In another embodiment, the treatment is epinephrine, and the parameters are dose and time between doses. In an embodiment, the dose is between 0.5 and 1.5 milligrams, and the time between doses is between 3 and 5 minutes. These treatment parameters are recommended by the AMERICAN HEART ASSOCIATION (AHA) for treating cardiac arrest. Of course, these parameters may be altered based on patient biological information, patient response to treatment, and so forth.

It should be understood that the use of “and/or” is defined inclusively such that the term “a and/or b” should be read to include the sets: “a and b,” “a or b,” “a,” “b.”

### BRIEF DESCRIPTION OF THE DRAWINGS

FIG. 1 shows a medication dosage timer of the cardiac response interface of an embodiment of the disclosed technology.

FIG. 2 shows a patient information database of the cardiac response interface of an embodiment of the disclosed technology.

FIG. 3 shows an active treatment log of the cardiac response interface of an embodiment of the disclosed technology.

FIG. 4 shows a flow chart of a method of carrying out an exemplary cardiac emergency response in accordance with an embodiment of the disclosed technology.

FIG. 5 shows a timeline of dosage intervals for different treatments administered during a cardiac emergency in accordance with an embodiment of the disclosed technology.

FIG. 6 is a high level block diagram of a node that may be used to carry out an embodiment of the disclosed technology.

### DETAILED DESCRIPTION OF EMBODIMENTS OF THE DISCLOSED TECHNOLOGY

In an embodiment of the disclosed technology, a method and system are used for assisting and facilitating cardiac emergency treatment. The method employs an electronic interface for logging the different treatments and/or medicines administered to a cardiac emergency patient. The interface maintains numerous time intervals, dosage frequencies, and dosage amounts for different treatments. The interface prompts the user when to administer different treatments based on the type of the ongoing cardiac emergency, as well other factors. The device implementing the electronic interface may further be in communication with treatment mechanisms, such as a defibrillator and/or an automatic dosage administration machine, for purposes of automated application of treatment at the prompts. Patient information and history, including the history from the present treatment regimen, is stored in a database accessible via the interface. The interface may be available locally on any electronic device, such as, for example, a smart phone, tablet, laptop, desktop, PDA, e-reader, etc. The software may run on any operating system, such as, for example, Windows (all versions), OSX, Linux, Unix, Mac, Android, and iOS. Further, the interface may be stored and managed from a remote server, accessible by any device with a network connection.

Embodiments of the disclosed technology will become clearer in view of the following description of the drawings.

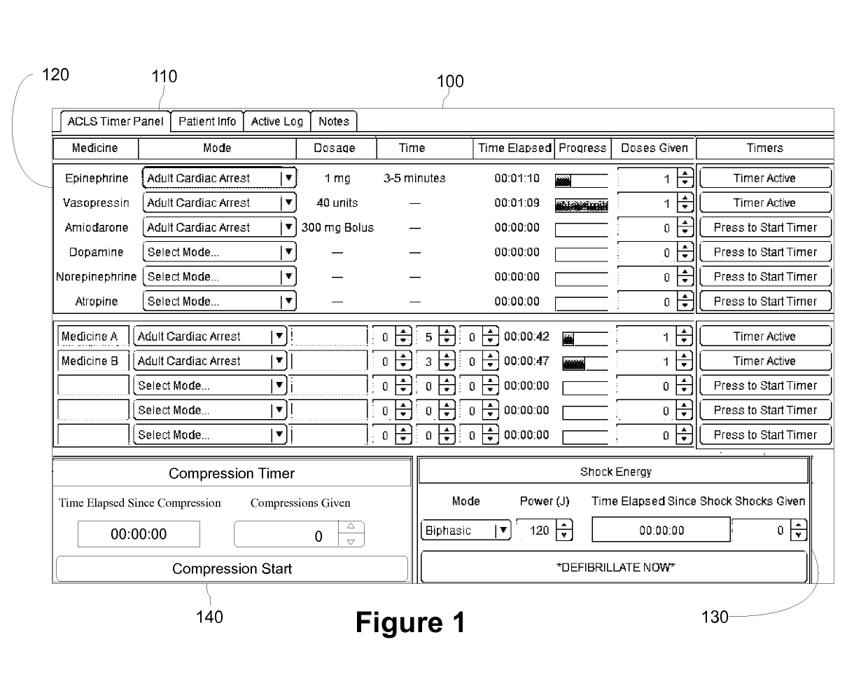

FIG. 1 shows a medication dosage timer of the cardiac response interface of an embodiment of the disclosed technology. The electronic interface 100 (herein “interface 100”) may be carried out on any electronic device having a display, memory and a processor. The interface 100 and associated methodology may be stored on the memory and executed by the processor. Alternatively, the interface 100 may be stored remotely at, and accessed via, a network node. In this embodiment, the device may be connected to any type of wireless network, such as, for example, a local area network (LAN), a wide area network (WAN), a mobile phone network, a packet switch data network, radio waves, or any other means for wirelessly sending information. All interactions between a user and the interface 100 may be exchanged via the network. “User,” for purposes of this specification, is defined as the person who is administering treatment. That is, the user may be a doctor, nurse, medical technician, emergency medical responder, police, etc. The “patient,” for purposes of this specification, is the individual who is suffering the cardiac emergency (i.e., the person being treated). It is possible, in rare instances, that the “user” may also be the “patient” if an individual is treating him/herself in the case of a minor cardiac condition occurring in the absence of other people.

The interface 100 is displayed on a screen or display of the device. An input device or touch screen may be used to manipulate and interact with the interface 100. Referring still to FIG. 1, the interface 100 has an Advanced Cardiovascular Life Support (“ACLS”) timer panel 110. The timer panel 110 is the portion of the interface which prompts a user to administer different treatments based on a number of identified factors. A medicine treatment window 120 lists different medicines and treatment regimens. The medicines shown are epinephrine, vasopressin, amiodarone, dopamine, norepinephrine, and atropine; each of which is known to be used in the treatment of different cardiac conditions. The “Mode” column has drag-down menus for user input of the type of cardiac emergency being experienced. In FIG. 1, ‘Adult Cardiac Arrest’ is selected, thereby activating a pre-specified dosage and time interval regimen for administering each medicine. Thus, a user who is not familiar with treating a cardiac emergency may simply follow the prompts in administering treatment. Because different cardiac conditions are treated with different treatment regimens, a user need only identify what the patient is experiencing and input a mode which corresponds thereto. Once inputted, pre-determined, ACLS recommended dosage amounts and intervals may be displayed and initiated. Each time a dose is administered, the user may record the dose, permitting the time to be reset. In addition to the common cardiac emergency medicines, the user may input other medicines listed below. In FIG. 1, ‘Medicine A’ is entered for treatment of ‘Adult Cardiac Arrest.’ The user-inputted time interval is five minutes in this example. Thus, dosing of ‘Medicine A’ is also tracked via the interface. Upon entering a time interval, the user may start the timer under the ‘Timers’ column.

In a further embodiment, the device on which the interface 100 is employed may be connected to, or in communication with, an automated dosage machine or intra-venous dosing apparatus. As such, the interface 100 may cause the doses of medicine to be administered automatically via the dosing apparatus.

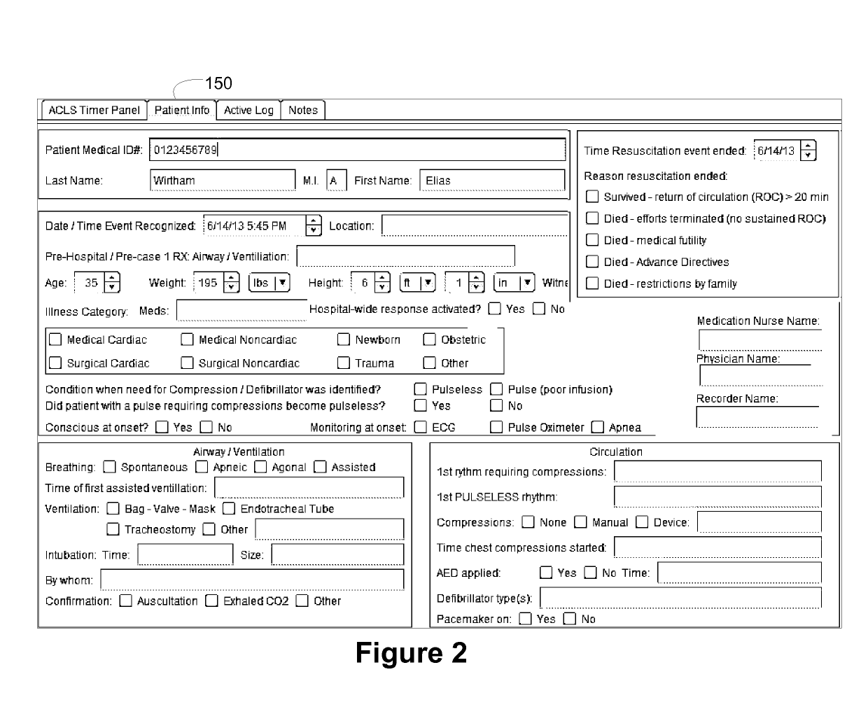

A patient reference window (not shown) may also by displayed on the interface 100. This window shows the relevant biographical data about the patient. This information may be inputted manually (see FIG. 2) or already stored within the database. The information in this window may dictate the dosage amounts and time intervals used on the patient.

Referring still to FIG. 1, a defibrillation window 130 is provided within the interface 100. The defibrillation window 130 allows the user to manually input desired defibrillation mode and power. Alternatively, ACLS recommended defibrillation parameters may be used based on the type of cardiac emergency and the biological details of the patient. The defibrillation prompts the user when to defibrillate based on this information. The user inputs and tracks the number of shocks administered in the defibrillation window 130. In a further embodiment, the device on which the interface 100 is employed may be connected to, or placed in communication with, an automated or semi-automated external defibrillator. The interface 100 may automatically cause the defibrillator to apply shocks to the patient according to the specified parameters.

Further, a compression timer window 140 may also be provided within the interface 100. The compression timer window 140 tracks the cardiopulmonary resuscitation (“CPR”) compressions applied to the patient. The compression timer window 140 also receives an input from the user to track the time elapsed from when compressions were given. This timer may have a fixed two minute limit. Once the two minutes have expired, the program alerts the user (voice) of compression timer expiration. In another embodiment, the timing frequency of the compression timer window 140 may vary based on the patient's biological information as well as the inputted classification of cardiac emergency.

FIG. 2 shows a patient information database of the cardiac response interface of an embodiment of the disclosed technology. A patient information tab 150 stores all historical and biographical medical information regarding the patient currently being treated. The data found or entered in patient information tab 150 is skewed towards a cardiac patient, in that much of the information pertains to cardiac history and conditions. Furthermore, the information inputted in the patient information tab 150 may be employed by the timer panel 110 in determining which treatments, medicines, doses and/or time intervals to use on a given patient. For example, a patient weighing 250 pounds may require a higher dosage and/or defibrillation power than a patient weighing 120 pounds.

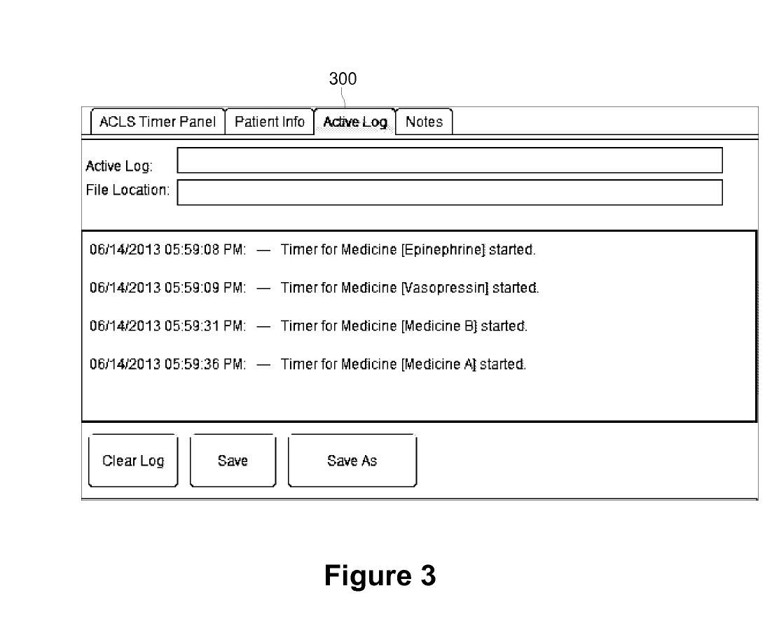

FIG. 3 shows an active treatment log of the cardiac response interface of an embodiment of the disclosed technology. The treatment log 300 displays a text list of all the medicines, doses and other treatments administered to the patient. The list is stored in the interface and/or the database and may be used in administering future treatments to the patient.

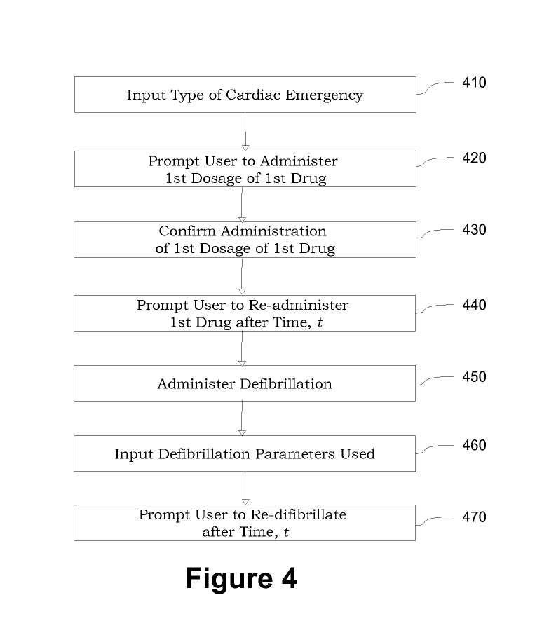

FIG. 4 shows a flow chart of a method of carrying out an exemplary cardiac emergency response in accordance with an embodiment of the disclosed technology. The method is an example of a typical initiation of a treatment regimen, the administration of which may be facilitated by the interface 100 of FIGS. 1 through 3. The method begins in step 410 when the user inputs the type of cardiac emergency into the interface. Of course, before this step, the user will have to access or display the interface on a device. Such may be done by executing a software application or operating system on the device, or accessing a network node via a network connection of the device (e.g., via a web browser).

In step 420, the user is prompted to administer a first dosage of a first drug. Upon administration, the user inputs a confirmation of having administered the drug in step 430. This begins the running of a timer pertaining to the first drug. The timer is set to operate on a time interval recognized to be optimal by the interface or the ACLS. After the time interval, t, has elapsed for the first drug, the user is prompted in step 440 to re-administer the first drug. The value for t is dictated by the type of cardiac emergency, the type of medicine being administered, and any other relevant biological/historical factors. This loop may continue perpetually until treatment of the cardiac emergency is stopped.

In step 450, initial defibrillation is administered to the patient. In manual defibrillation mode, the defibrillation parameters are inputted via the defibrillation window in step 460. These parameters are used by the interface to determine when the next shock should be applied. Thus, in step 470 the user is prompted to re-defibrillate after time, t. The steps carried out in the example shown in FIG. 4 are simple and merely for explanatory purposes. In the field, a multitude of medicines/treatments and dosages/frequencies may be employed during a single treatment of a single cardiac patient. Keeping track of all of these steps may be difficult for a user under emergency conditions, thus the interface of the disclosed technology logs and keeps track of which steps should be carried out and when.

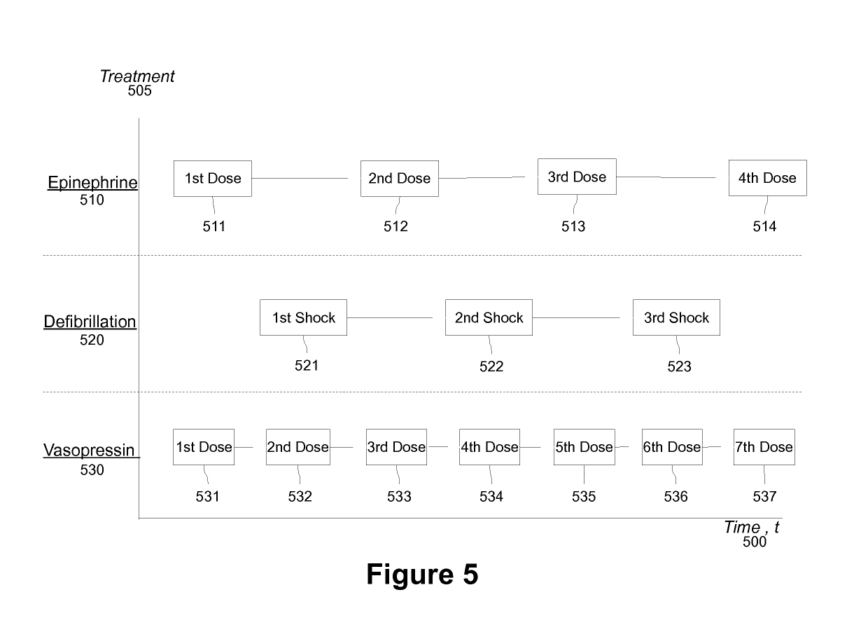

FIG. 5 shows a timeline of dosage intervals for different treatments administered during a cardiac emergency, in accordance with an embodiment of the disclosed technology. Such would be a visualization of an active treatment log prescribed by an embodiment of the disclosed technology. In the example shown, three different treatments 505 are used, each having its own treatment regimen. The treatments are epinephrine 510, defibrillation 520, and vasopressin 530. The treatments 505 are shown as being applied over time, t 500. The first treatment, epinephrine 510, is depicted as being administered in four doses 511-514. The first dose 511 is applied, and subsequent doses 512, 513 and 514 are administered after the passage of a specified time interval. In the example shown, the second treatment, defibrillation 520, is first administered in between the first dose 511 and the second dose 512 of epinephrine 510. Thus, the first shock 521 is applied, followed, after a specified time interval, by a second shock 522 and a third shock 523.

The third treatment depicted in FIG. 5 is vasopressin 530. For purposes of this example, vasopressin 530 has a shorter time interval between doses. Thus, in the same total time interval that four doses of epinephrine 510 and three defibrillator shocks are administered, seven doses of vasopressin 530 are applied. As such, a first dose 531 of vasopressin 530 is applied at about the same time as the first dose 511 of epinephrine 510. After a time interval, a second dose 532 is administered. Each of the dose administrations is logged in the interface by the user. The interface uses the entered times/doses to determine when the next dose should be applied, in order to prompt the user. More than three treatments may be used, and many doses may be administered and logged; the timeline shown in FIG. 5 is merely an example of a possible sequence of steps carried out to initialize treatment on a patient.

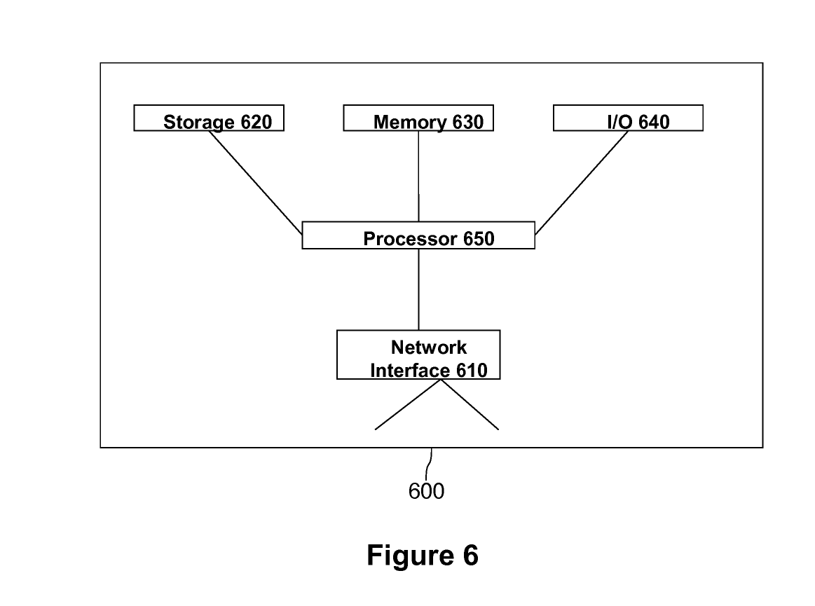

FIG. 6 is a high level block diagram of a node that may be used to carry out an embodiment of the disclosed technology. Device 600 comprises a processor 650 that controls the overall operation of the computer by executing the device's program instructions which define such operation. The device's program instructions may be stored in a storage device 620 (e.g., magnetic disk, database) and loaded into memory 630 when execution of the console's program instructions is desired.

Thus, the device's operation will be defined by the device's program instructions stored in memory 630 and/or storage 620, and the console will be controlled by processor 650 executing the console's program instructions. A device 600 also includes one, or a plurality of, input network interfaces for communicating with other devices via a network (e.g., radio signaling). The device 600 further includes an electrical input interface for receiving power and data from a battery or other power source.

A device 600 also includes one or more output network interfaces 610 for communicating with other devices. Device 600 also includes input/output 640 representing devices which allow for user interaction with a computer (e.g., display, keyboard, mouse, speakers, buttons, etc.). One skilled in the art will recognize that an implementation of an actual device will contain other components as well, and that FIG. 6 is a high level representation of some of the components of such a device for illustrative purposes. It should also be understood by one skilled in the art that the methods and systems depicted in FIGS. 1 through 5 may be implemented on a device such as is shown in FIG. 6.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods and apparatuses described hereinabove are also contemplated and within the scope of the invention.

I claim:

1. A method for facilitating cardiac emergency treatment comprising:
receiving an input from a user classifying the type of cardiac emergency being experienced by a patient;
prompting said user to administer said patient with a first dosage of a first drug based on the inputted cardiac emergency classification;
receiving an input confirming the administration of said first dosage of said first drugs;
prompting said user to re-administer said first drug after a pre-specified time has elapsed;
receiving an input from said user about defibrillation parameters used in a first defibrillation applied to said patient, said defibrillation parameters being defibrillation mode and amount of power;
prompting said user to re-defibrillate after a specific time interval has elapsed;
receiving an input from said user when a compression has been applied to said patient; and
prompting said user to apply another compression after a compression time interval has elapsed.

2. The method of claim 1, further comprising a step of receiving input information regarding patient characteristics, and using said patient characteristics to determine treatment parameters.

3. The method of claim 2, wherein said patient characteristics comprise age, weight, height, and medications.

4. The method of claim 1, wherein said defibrillator is connected to said interface, and said interface triggers each defibrillation at said specified time intervals.

5. The method of claim 1, further comprising a step of logging all data inputted.

6. The method of claim 5, wherein said data inputted is used for determining future treatment regimens.

7. The method of claim 1, wherein said specific time interval is based on said inputted cardiac emergency classification and said defibrillation parameters.

8. The method of claim 1, wherein said specific time interval is two minutes.

9. The method of claim 1, wherein said compression time interval is two minutes.

10. The method of claim 1, wherein said compression time interval is based on said inputted cardiac emergency classification and said defibrillation parameters.

11. A system for using artificial intelligence to facilitate treatment on a patient experiencing a cardiac emergency, comprising:
a data processing apparatus;
a computer storage device storing instructions that, when executed by data processing apparatus, cause the data processing apparatus to perform operations;
an interactive interface for inputting real-time patient data and displaying treatment prompts, based on said real-time patient data;
a database for logging patient data and treatments administered; and
artificial intelligence to modify treatment prompts based on inputted real-time patient data.

12. The system of claim 11, wherein said inputted patient data are received electronically from sensors associated with said patient.

13. The system of claim 11, further comprising a defibrillator electronically coupled to said data-processing apparatus, wherein said defibrillator is controlled by said interface.

14. The system of claim 11, further comprising an automated dosing machine electronically coupled to said data-processing apparatus for administering medicine to said patient via said interface.

15. A method of using artificial intelligence for determining and prescribing a treatment regimen for a patient suffering from a cardiac emergency, comprising:
receiving inputted symptom data observed from said patient;
receiving inputted biological data regarding said patient;
diagnosing said cardiac emergency suffered by said patient using artificial intelligence;
prescribing a protocol for treating said patient; and
displaying, on a device, prompts outlining parameters for a treatment to be administered to said patient;
receiving and logging inputted treatment data regarding said treatment administered to said patient; and
modifying said parameters based on said inputted treatment data.

16. The method of claim 15, wherein said treatment is defibrillation.

17. The method of claim 16, wherein said parameters comprise defibrillation mode, power and time between shocks.

18. The method of claim 15, wherein said treatment is epinephrine.

19. The method of claim 18, wherein said parameters are dose and time between doses.

20. The method of claim 19, wherein:
said dose is between 0.5 and 1.5 milligrams; and
said time between doses is between 3 and 5 minutes.
